(19) United States
(12) Patent Application Publication
(10) Pub. No.: US 2013/0165943 A1
(43) Pub. Date: Jun. 27, 2013

(54) INTRAOCULAR LENS SURGICAL SYSTEM AND METHOD

(71) Applicant: David A. Downer, Fort Worth, TX (US)

(72) Inventor: David A. Downer, Fort Worth, TX (US)

(21) Appl. No.: 13/617,979

(22) Filed: Sep. 14, 2012

Related U.S. Application Data
(60) Provisional application No. 61/579,870, filed on Dec. 23, 2011.

(51) Int. Cl. A61F 9/00

(52) U.S. Cl. 606/107

(57) ABSTRACT

Various systems, apparatuses, and processes may be used for performing intraocular lens surgery. In some implementations, a system for intraocular lens surgery may include a first body, a second body, and flexible joint. The first body may be adapted to receive an intraocular lens, and the second body may include a passage adapted to facilitate folding the intraocular lens as the lens is advanced through the passage. The flexible joint may be interposed in a gap between the first body and the second body.

### RELATED APPLICATIONS

This application claims priority to U.S. provisional application Ser. No. 61/579,870, filed on Dec. 23, 2011, the contents which are incorporated herein by reference.

### BACKGROUND

The present disclosure relates to optical surgery, and more specifically to surgery for replacement of a patient's lens.

The human eye, in simple terms, functions to provide vision by transmitting and refracting light through a clear outer portion called the cornea and focusing the image by way of the lens onto the retina at the back of the eye. The quality of the focused image depends on many factors including the size, shape, and length of the eye, and the shape and transparency of the cornea and lens.

When trauma, age, or disease causes the lens to become less transparent, vision deteriorates because of a reduction in light transmitted to the retina. This deficiency in the eye's lens is medically known as a cataract. The treatment for this condition is often surgical removal of the lens and implantation of an artificial lens, often termed an intraocular lens (IOL).

An IOL is often foldable and inserted into the eye through a relatively small incision by being advanced through an insertion cartridge, which causes the IOL to fold. The IOL is typically advanced through the insertion cartridge by a plunger-like device.

### BRIEF SUMMARY

In one general implementation, a system for intraocular lens surgery may include a first body adapted to receive an intraocular lens and a second body including passage adapted to facilitate folding the intraocular lens as the lens is advanced through the passage. The system may also include a flexible joint interposed in a gap between the first body and the second body. In particular implementations, the second body is composed of a fluoropolymer.

In some implementations, the first body is adapted to facilitate static folding of the intraocular lens. In certain implementations, the static folding of the first body does not materially distort the second body. The first body may, for example, be composed of a relatively flexible material compared to the second body. In particular implementations, the first body may include an open channel, with a first tab coupled to one end of the open channel and a second tab coupled to the second end of the open channel.

The flexible joint may, for example, include a flexible material molded over the first body and the second body. In certain implementations, the flexible material is continuous from the first body to the second body.

Various implementations may include one or more features. For example, a system for intraocular lens surgery may provide a relatively large static folding of an IOL without distorting body 120. Thus, increased folding of an IOL may be achieved without affecting performance of system 100. Moreover, an IOL may be folded to a relatively small size (e.g., >500%) due to the static folding and dynamic folding. As a further example, an IOL lens may be moved from one body (e.g., one that statically folds the IOL) to another body (e.g., one that dynamically folds the IOL) with reduced damage (e.g., snagging, scuffing, and/or scratching) of the IOL, which allows the IOL to be more consistently delivered in a usable state. As a further example, in particular implementations, low coefficient of friction materials (e.g., fluoropolymers) may be used without requiring a lubricating agent.

The details and features of various implementations will be conveyed by the following description, along with the drawings.

### BRIEF DESCRIPTION OF THE SEVERAL VIEWS OF THE DRAWINGS

FIGS. 1A-B illustrate an example system for intraocular lens surgery.

FIGS. 2-6 show example enhanced bonding features for joining a first body, a second body, and a flexible joint.

FIG. 7 shows an example intraocular lens insertion tool for use with an intraocular lens surgery system.

FIGS. 8-11 show cross-sectional views showing example closed configurations of example systems for intraocular lens surgery.

FIG. 12 is a flowchart illustrating an example process for using a system for intraocular lens surgery.

### DETAILED DESCRIPTION

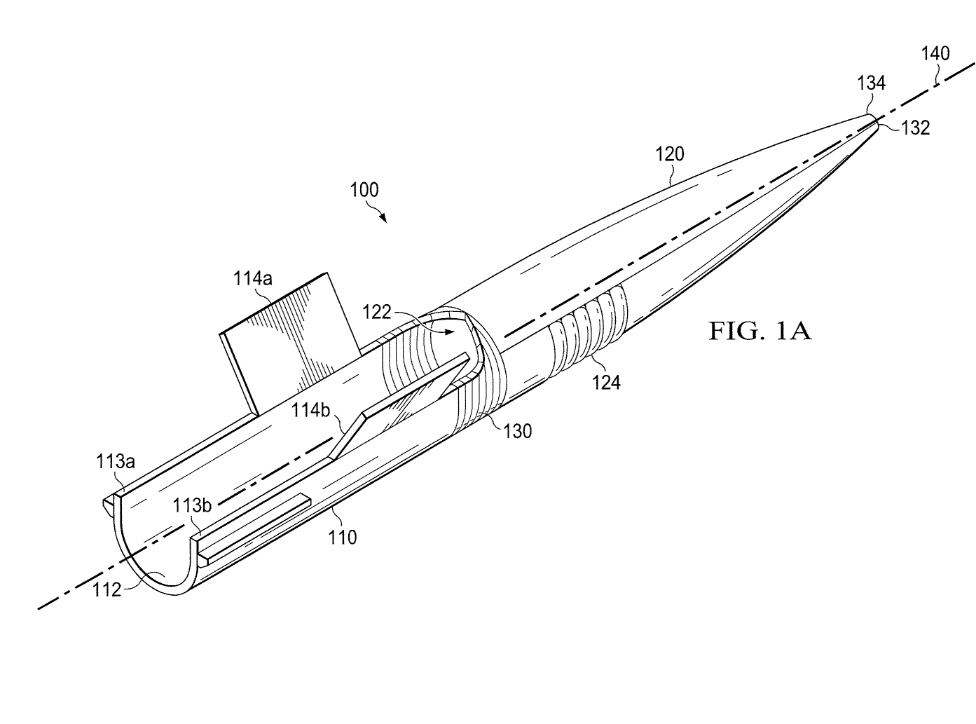

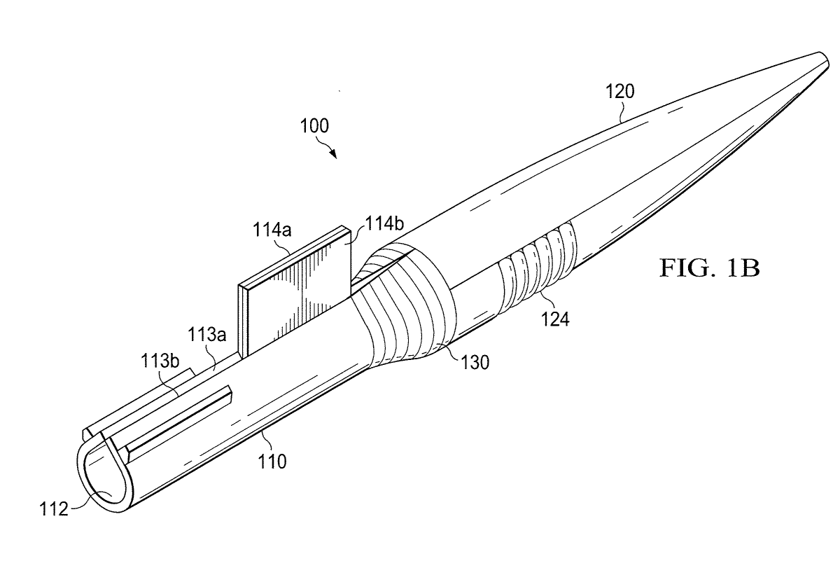

FIGS. 1A-B illustrate an example system 100 for use in ophthalmic surgery. Particularly, system 100 may be used in intraocular lens surgery. System 100 includes a first body 110, a second body 120, and a flexible joint 130, which is located between first body 110 and second body 120. FIG. 1A illustrates system 100 in a first state in which the first body 110 is in an open configuration. FIG. 1B illustrates system 100 in a second state in which the first body 110 is in a closed configuration.

First body 110 includes an open channel 112 adapted to receive an IOL. In some instances, an IOL may, for example, be approximately 6 mm in diameter. Taking the haptics of an IOL into account, the IOL may have a diameter up to approximately 13 mm, for example. In some implementations, the cross section of open channel 112 may be u-shaped, as shown in FIG. 1A. In other implementations, the cross section of open channel 112 may have other shapes. For instance, the cross section may be c-shaped. Open channel 112 includes a first end 113a and a second end 113b.

The system 100 may also include tabs 114a, 114b. FIG. 1A shows the tabs 114a, 114b in an open configuration in which the channel 112 is open to receive an IOL. FIG. 1B shows the tabs 114a, 114b in a closed configuration in which the channel 112 is closed. Tabs 114a, 114b may be coupled to ends 113a, 113b, respectively. Tabs 114a, 114b may be sized to allow a user (e.g., a physician or other medical professional) to manually engage the tabs and move them towards each other, such as by squeezing the tabs 114a, 114b together. For example, in some instances, the tabs 114a, 114b may be manually engaged by the fingers of the user or via another device. Tabs 114a, 114b may define an angle therebetween. The angle may be any desired angle. For example, in some implementations, the angle between tabs 114a, 114b may be between 20 and 180 degrees. In the illustrated implementation, tabs 114a, 114b are integrally formed to body 112 (e.g., they are molded at the same time as the body). However, in other implementations, tabs 114a, 114b may be otherwise coupled to first body 110. For example, the tabs 114a, 114b may be coupled to the first body 110 with an adhesive, a fastener, or in any other suitable way.

The first body 110 is operable to fold and, in some instances, compress an IOL inserted into the channel 112. For example, in some instances, an IOL inserted into channel 112 may be predominantly folded and, to a small amount, compressed as a result of the bringing the tabs 114a, 114b close together. Thus, in some implementations, a function of the first body 110 is primarily to fold the IOL with little to any compression thereof in preparation for further folding and compression within the second body 120. However, in other implementations, the first body 110 may be utilized to perform both initial folding of the IOL and provide increased amounts of compression of the IOL. That is, in some implementations, the first body 110 may provide an IOL compression in addition to providing an initial folding of the IOL.

In some implementations, first body 110 may be formed from a relatively flexible material. For example, first body 110 may be formed from polyurethane, polyether block amide (PEBA), polyethylene, Pebax® produced by Arkema, Inc., 900 First Avenue, King of Prussia, Pa. 19406, or any other suitable material. However, first body 110 may be formed from any suitable material.

The second body 120 includes a passage 122 extending through the second body 120. Passage 122 may have a symmetric or asymmetric bore and generally tapers along its length to assist in folding an IOL. The passage 122 forms an outlet 132 at an end thereof. In some instances, the outlet 132 may form an oval or elliptical shape. In other instances, the outlet 132 may have a circular shape. However, the outlet 132 may have any suitable shape. For example, in some instances, the outlet 132 may have an asymmetric shape.

The second body 120 is adapted to fold and compress an IOL as the IOL is advanced through the passage 122. For example, in some implementations, the passage 122 is operable to at least one of fold and/or compress an IOL passing therethrough. For example, in some instances, the passage 122 may have a tapered shape. The tapered shape may be operable to fold the IOL along at least a portion of the length of the passage 122 as well as compress the IOL. In some implementations, as the IOL is advanced through the passage 122, both folding and compression of the IOL is increased. Additionally, in some instances, as the IOL is advanced through the passage 122, the rate of folding of the IOL (that is, the amount of folding of the IOL per length of the passage 122) decreases. Thus, in some instances, the IOL reaches a certain point along the length of the passage 122 where most if not all of the manipulation of the IOL is compression so as to reduce the size of the IOL prior to insertion into the eye.

The second body 120 may include one or more tactile units 124. A tactile unit 124 may be provided on one or more surfaces of the second body 120. For example, tactile units 124 may be provided on opposite sides of the exterior surface of the second body 120 to enhance tactile feedback to a user. In other instances, tactile unit 124 may form a continuous ring around all or a portion of the external surface of the second body 120 or other portion of system 100. Thus, tactile units 124 may assist in the grasping, whether by a user's hand or a tool, of second body 120, and system 100 accordingly.

During use, a foldable IOL, which may be made of silicone, soft acrylics, hydrogels, or other suitable materials, is advanced through passage 122 in preparation for insertion into the eye. As mentioned previously, an IOL having haptics may be approximately 13 mm in diameter with the haptics taken into account. However, surgical incisions are typically much smaller (e.g., 0.5-3 mm in width). An IOL is therefore typically folded before insertion through the incision formed in the eye. As an IOL is advanced through passage 122, the IOL is folded due to the shape of the passage. Folding of the IOL as it is advanced through the passage 122 is referred to as “dynamic folding”. Tip 134 of the system 100 may be inserted through an incision in the eye. The folded IOL is evacuated from the passage 122 via the outlet 132. Thus, in operation, the IOL may be inserted into the eye through the outlet 132.

In some instances, the second body 120 may be made of a relatively rigid material. For instance, second body 120 may be made of polypropylene. In certain implementations, second body 120 may also have a relatively low coefficient of friction. For example, the second body could be made of a flouropolymer (e.g., Polytetrafluoroethylene (PTFE) or Perfluoroalkoxy (PFA)). While some example materials are identified, the second body 120 may be formed from other suitable materials. In some implementations, the second body may include a lubricity enhancing agent. Example lubricity enhancing agent may be a solvent or aqueous based hydrophilic material. For example, PVP (polyvinylpyrrolidone) or coatings 2-TS-96 or 2314-172 produced by Hydromer, Inc. of 35 Industrial Parkway, Branchburg, N.J., may be used as lubricity enhancing agents.

As indicated above, the system 100 may also include a flexible joint 130. A flexible joint 130 may be interposed in one or more gaps between first body 110 and second body 120. Due to its flexibility, flexible joint 130 allows first body 110 to move relative to second body 120. Further, the flexible joint 130 may also provide a continuous surface between the inner surface of first body 120 and the inner surface of second body 130. That is, the inner surface of the flexible joint 130 provide a smooth, continuous transition between the channel 112 of first body 110 and the passage 122 of the second body 120. As a result, the IOL is made to easily pass through the system 100 without having to encounter a step or discontinuity within the system 100 that could impede passage of or damage the IOL. Further, the smooth transition provides for a more consistent force needed to advance an IOL through the system 100.

Flexible joint 130 may be made of any suitable flexible material. For example, in some implementations, flexible joint 130 may be formed from a thermoplastic elastomer. During manufacturing, the elastomer may, for instance, be overmolded onto first body 120 and second body 120 while their respective core pins are still engaged. In other implementations, flexible joint 130 may be made of silicone or other appropriate flexible material.

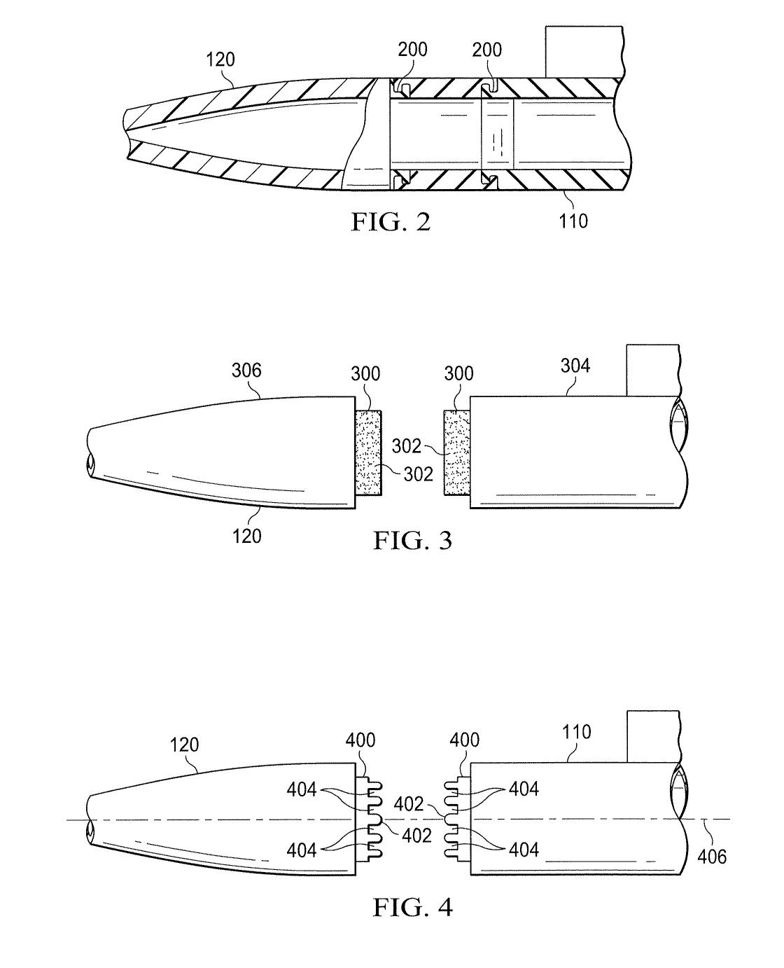

First body 110 and second body 120 may contain geometries to enhance mechanical bonding therebetween. For example, as shown in FIG. 2, the first body 110 and second body 120 may include annular grooves 200. In some instances, the flexible joint 130 may be molded separately and include corresponding ridges that are received into the annular grooves 200 when the flexible joint 130 is assembled to the first body 110 and second body 120. In other implementations, the flexible joint 130 may be formed directly onto the first and second bodies 110, 120 (also referred to as “overmolded”) resulting in interlocking between the flexible joint and the first and second bodies 110, 120 as a result of the annular grooves 200.

FIG. 3 shows another example geometry for enhancing bonding between the flexible joint 130 and the first and second bodies 110, 120. As shown in FIG. 3, the first and second bodies 110, 120 may include an extensions 300 having an outer surfaces 302 that are reduced (i.e., offset inwardly) from the outer surfaces 304, 306 of the first body 110 and second body 120, respectively. The outer surfaces 302 may be roughened or textured. As such, the roughened or textured outer surfaces 302 provide for improved mechanical interlocking with corresponding surfaces of the flexible joint 130 (not shown). For example, the roughened or textured outer surfaces 302 provide enhanced bonding when the flexible joint 130 is overmolded onto the first and second bodies 110, 120

FIG. 4 shows another example geometry. An end surface 402 of reduced extensions 400 may define a plurality of notches formed therein. In some instances, the plurality of notches 404 may be radially disposed about longitudinal axis 406. The plurality of notches 404 provide enhanced interlocking between the first and second bodies 110, 120 and the flexible joint 130. For example, bonding between the components is particularly enhanced when the flexible joint 130 is overmolded. When overmolded, for example, material forming the flexible joint 130 is received into and solidifies within the grooves 404 providing improved boding. Other methods of assembling the first body 110, second body 120, and flexible joint 130 may also be used.

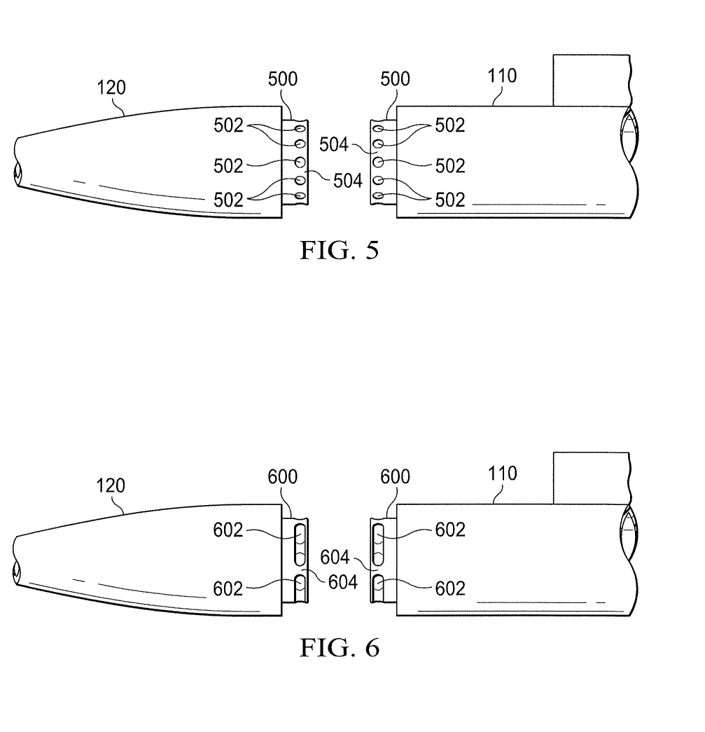

The enhanced bonding features illustrated in FIG. 5 include a plurality of openings 502 formed in outer surface 504 of reduced extensions 500. In some instances, the openings 502 may be through openings that define a plurality of perforations. In other instances, the plurality of openings 502 defines a plurality of recesses that do not extend entirely through the extensions 500. In still other instances, some of the openings 502 may fully extend through the extensions 500 while other openings may form recesses. That is some of the openings 502 may extend fully through the extensions 500 while other openings 502 may not fully extend through the extensions 500. FIG. 6 shows the enhanced bonding features to include a plurality of slots 602 formed in outer surfaces 604 of reduced extensions 600. In some instances, some of the slots 602 may extend fully through the extensions 600 while others may extend only partially through the extensions 600.

In a molding operation, such as overmolding, material forming the flexible joint 103 fills the openings 502 or slots 602 such that, after solidification of the flexible joint 103, the openings 502 and slots 602 create interlocking features to retain the flexible joint 103 to the first body 110 and the second body 120.

Although overmolding is particularly described with respect to the enhanced bonding geometries described herein, other types of construction between the first body 110, second body 120, and flexible joint 130 are within the scope of the disclosure. For example, the flexible joint 130 may be formed separately and include mating features to the enhanced bonding geometries included with the first body 110 and the second body 120 to form the system 100. Further, the enhanced bonding features may include interference fits to join the components. Still further, an adhesive may be used in combination with one or more of the enhanced bonding features to enhance bonding among the components.

In some implementations, second body may be a Monarch®-type or Acrysert®-type injection cartridge, both of which are produced by Alcon Laboratories, Inc. of Fort Worth, Tex. USA. However, these are provided only as examples. Thus, in other instances, other types of injection cartridges could also be used. For example, other commercially available or non-commercially available injection cartridges may be used.

Second body 120 also includes tactile units 124 (only one of which is visible).

In some implementations, system 100 may be approximately 60 mm in length. However, system 100 may have any desired size. For example, a desired application may affect the size of system 100. Additionally, the outlet 132 of the second body 120 may be a size selected to correspond to a particular incision size. For example, in some instances, the outlet 132 may have a diameter of approximately 0.8 mm. However, this size is not intended to be limiting. Rather, the outlet 132 may have any desired size. Further, while the size of the outlet 132 is discussed in terms of “diameter”, as mentioned above, the shape of outlet 132 may be other than circular. Thus, the size and/or shape of the outlet 132 may be selected, for example, to correspond to a size and/or configuration of an incision, an injection location, or other consideration.

An IOL insertion tool may be inserted into open channel 112 to engage the IOL and advance it towards second body 120. For example, an insertion tool, such as insertion tool 200 (discussed in more detail below), may be utilized to advance the IOL through channel 112 and passageway 122. While being advanced towards second body 120, the IOL crosses flexible joint 130.

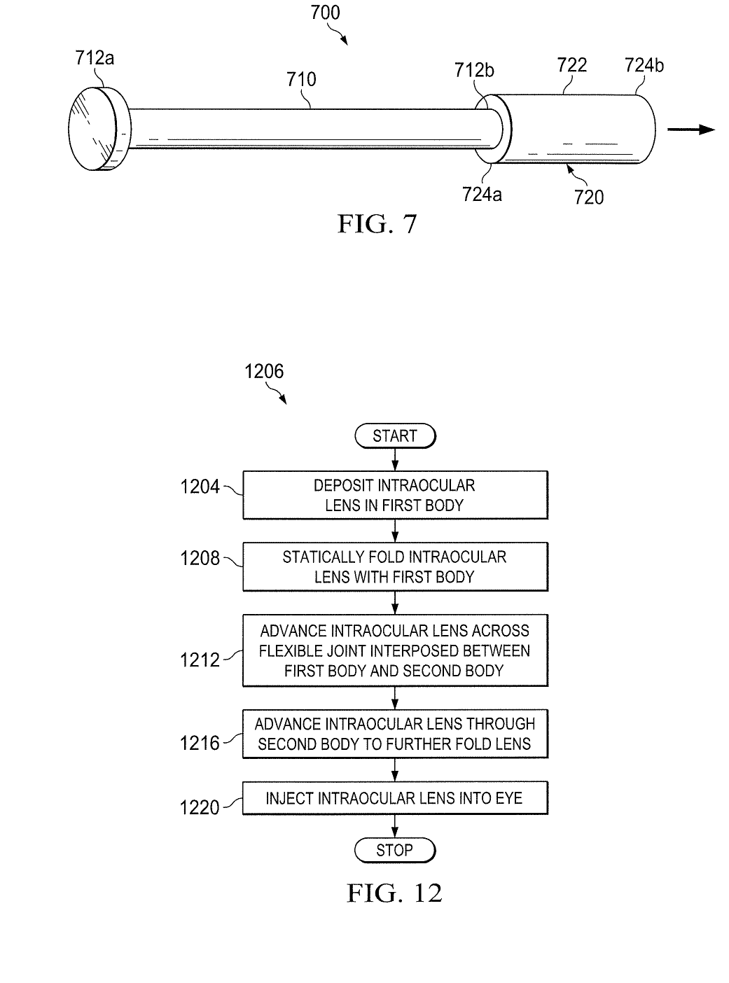

FIG. 7 illustrates an example IOL insertion tool 700. IOL insertion tool 700 includes an elongate body 710 having a first end 712a and a second end 712b. As illustrated, body 710 is in the form of a rod. However, the body 710 may have other shapes. Second end 712b of body 710 includes an IOL interface 720. IOL interface 720 is adapted to interface with an IOL and to advance the IOL through the system 100. In the illustrated implementation, IOL interface 720 includes a body 722 having a generally cylindrical shape. However, in other implementations, the body 722 may have other shapes. For example, in some instances, the body 722 may have an elliptical or oval cross-sectional shape. Further, in some instances, the shape of the body 722 may correspond to the shape of the channel 112 and/or passage 122. Other shapes are also within the scope of the disclosure.

The body 722 also includes a first end 724a and a second end 724b. In some instances, IOL interface 720 may be integral with body 710. In other instances, IOL interface 720 may be coupled to the body 710. For example, in some implementations, first end 724a may include a port into which second end 712b of the body 722, or a portion thereof, may be inserted. For example, the second end 712b may include a protrusion that is received within a receptacle formed in the body 710. In some instances, body 710 and the IOL interface 720 may form an interference fit. Second end 724b may be closed.

In some implementations, body 710 may be formed from a metal. For example, body 710 may be formed from stainless steel or titanium. In other implementations, body 710 may be formed from a polymeric material. For example, body 710 may be formed from polyurethane, polypropylene, styrene, or a commercial injection-molded elastomer. Further, the body 710 may be formed from any other suitable material. In some implementations, IOL interface 720 may be approximately 1 to 3 mm in diameter. Also, while the IOL interface 720 is discussed in terms of diameter, as indicated above, IOL interface 720 may have cross-sectional shapes other than circular.

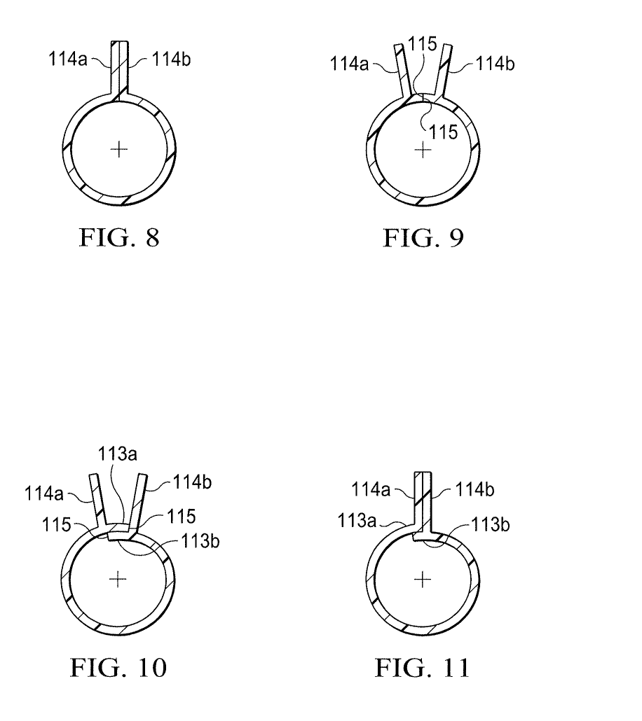

In certain modes of operation, an IOL is deposited into open channel 112. For example, the IOL may be received at the locations of tabs 114a, 114b. Then, tabs 114a, 114b may be moved towards each other. FIG. 1B illustrates the example system 100 with tabs 114a, 114b moved together in the closed configuration. FIG. 8 shows a cross section the example system 100 in which the tabs 114a, 114b are in substantial contact in the closed configuration.

However, in other implementations, the tabs 114a, 114b need not be in contact with each other in the closed configuration. That is, in some implementations, the tabs 114a, 114b may be arranged on the first body 110 such that, when the tabs 114a, 114b are in the closed configuration, a gap remains between tabs 114a, 114b. For example, FIG. 9 is a cross-sectional view of an example system in which the tabs 114a, 114b are offset when in the closed configuration. In still other implementations, an end 113a may overlap end 113b, as shown in FIG. 10. As also shown in FIG. 10, the tabs 114a, 114b are offset in the closed configuration. In still other implementations, ends 113a, 113b may overlap while the tabs 114a, 114b are brought into contact in the closed configuration, as shown in FIG. 11. Thus, the tabs 114a, 114b may have numerous configurations, as illustrated by the examples described herein.

Further, in still other implementations, when being placed in the closed configuration, one tab may be displaced a greater amount than the other tab. That is, the tabs may be displaced different amounts when placing the channel 112 in the closed configuration. Still further, in some implementations, both tabs 114a, 114b may be moved when placing the channel 112 in the closed configuration. Additionally, although FIGS. 9 and 10 show that the tabs 114a, 114b may be offset substantially the same amount from edges 115, the tabs 114a, 114b may be offset from the edges 115 by different amounts. In still other implementations, one tab may be offset from an edge 115 while the other tab may not be offset from the edge 115, as shown in FIG. 11, for example.

The movement of tabs 114a, 114b into the closed configuration causes the IOL to be folded and, in some instances, compressed without moving the IOL along the longitudinal axis of system 100. This type of folding is referred to as “static folding”. In other implementations, movement of the tabs 114a, 114b in the closed configuration may cause partial folding of the IOL.

As explained above, movement of the tabs 114a, 114b into the closed configuration causes channel 112 to close or substantially close. Thus, movement of the tabs 114a, 114b into the closed configuration causes distortion or a shape change of the first body 110. Further, the flexible joint 130 or a portion thereof may also be distorted as a result of movement of the tabs 114a, 114b into the closed configuration. For example, in placing the tabs 114a, 114b in the closed configuration, the first body 110 is distorted as a result of closing the channel 112. Further, distortion of the flexible joint 130 may vary along the length of the flexible joint 130. For example, the flexible joint 130 may distort a greater amount near the first body 110 and a lesser amount near the second body 120.

An insertion tool, such as insertion tool 200, may be inserted into the closed channel 112, and the IOL interface 220 may engage the folded IOL. The insertion tool 200 advances the IOL through the remainder of the closed channel 112 and over the flexible joint 130. The IOL crosses the flexible joint 130 and enters passage 122. The insertion tool 200 further advances the IOL through the passage 122. In some implementations, as the IOL is advanced through the passage 122, the IOL is dynamically folded (that is, folding occurring during movement of the IOL relative to the system 100) as a result of the contour of the passage 122. Additionally, the IOL is compressed as a result of travel through the passage 122. Thus, in some instances, movement of the IOL through the passage 122 may result in both additional folding and increased compression of the IOL. When the IOL reaches the end of the passage 122, the IOL is in a fully folded and compressed configuration and ready to be inserted into the eye.

System 100 may provide a variety of features. For example, because a relatively flexible material may be used for body 110 while a relatively rigid material may be used for body 120, system 100 may provide a relatively large amount of dynamic folding and/or compression of an IOL without distorting second body 120. Thus, movement of the IOL through the passage 122 causes little, if any, distortion of the second body 120. In some implementations, the second body 120 is substantially unchanged as a result of movement of the tabs 114a, 114b into the closed configuration. That is, the second body 120 experiences minimal distortion, if any, as a result of movement of the tabs 114a, 114b into the closed configuration. Thus, all or substantially all of the distortion experienced by the system 100 when tabs 114a, 114b are placed in the closed configuration occurs in the first body 110 and flexible joint 130. Further, the second body 120 undergoes little if any distortion as a result of passage of an IOL through passage 122. Thus, the second body 120 remains substantially unchanged as a result of passage of an IOL through passage 122.

Various aspects of the system 100 may be varied in order to prevent or substantially prevent distortion of the second body 120. For example, in some instances, an axial length of the flexible joint 130 may be varied in order to minimize or prevent distortion of the second body 120 as a result of manipulation of first body 110 (e.g., placing the tabs 114a, 114b into the closed configuration) and/or movement of an IOL through the passage 122. Also, a durometer value of the material used to form the second body 120 may be selected or varied in order to minimize or prevent distortion of the second body 120 as a result of manipulation of the first body 110 and/or movement of an IOL through passage 122. Further, a durometer value of the material used to form the flexible joint 130 may also be varied to control distortion of the second body 120.

Thus, increased folding and compression of an IOL may be achieved without affecting performance of system 100.

Moreover, an IOL may be folded to a relatively small size due to the static folding, dynamic folding, and compression of the IOL as it is placed into and passed through the system 100. For example, in some instances, the system 100 may reduce the size of an IOL up to 500 percent or more. That is, upon passing through the system 100, a fully folded IOL may have a reduction in size that is 500 percent or more than the initial, unfolded and uncompressed size of the IOL. In particular implementations, for example, the system is operable to fold and compress an IOL such that the folded and compressed IOL is insertable through an incision of less than 1 mm in width. For example, in some instances, the system 100 is operable to fold and compress an IOL such that the IOL is insertable through a 0.8 mm incision.

A benefit of the system of the present disclosure is that, in some implementations, low coefficient of friction materials, such as fluoropolymers, may be used without the use of lubricating agents. By avoiding the use of lubricating agents, manufacturing costs are reduced and hoop stresses formed in the second body associated with smaller bores as the IOL is advanced through the system are also reduced. Additionally, the smooth interface between the channel 112 and passage 122 provided by the flexible joint 130 substantially reduces or eliminates the risk of damage to the IOL as the IOL is advanced through the system 100. This is because the smooth transition substantially reduces or eliminates snagging, scuffing, and/or scratching of the IOL. As a result, the system 100 is operable to better deliver the IOL in a usable state.

Although FIGS. 1A-B illustrate one implementation of an IOL surgical system, other implementations may include fewer, additional, and/or a different arrangement of components. In some implementations, for example, tabs 114a, 114b may be the full length of body 110. Additionally, tabs 114a, 114b may be offset from each other along the longitudinal axis to allow further folding of the tabs, possibly creating an overfolding condition for first body 110. For example, in an overfolded condition, ends 113a, 113b may overlap each other. Tabs 114a, 114b may also be mounted to the circumference of open channel 112 to create an overfolding condition.

Additionally, in some implementations, tabs 114a, 114b may be movable by differing amounts around the longitudinal axis 140. This may assist in situating and/or static folding of an IOL. Moreover, in particular implementations, tabs 114a, 114b may move relative to each other in one or more directions other than movement about the longitudinal axis 140. For example, in some instances, the tabs 114a, 114b may also be movable axially relative to each other and twisted relative to each other. In certain situations, this may be advantageous for the situating and/or static folding of an IOL, such as within the first body 110.

FIG. 12 illustrates an example process 1200 for using a system for intraocular lens surgery. Process 1200 may, for instance, be performed using a system similar to system 100.

Process 1200 includes depositing an IOL in a first body of an intraocular lens surgical system (operation 1204) and statically folding the IOL with the first body (operation 1208). For instance, an IOL may be manually deposited into a body that may be folded radially about its longitudinal axis. In some instances, static folding may be done manually. For example, static folding may be accomplished when a user brings tabs, such as tabs 114a, 114b of the first body 110, together using the user's fingers or with the use of a tool.

Process 1200 also includes advancing the IOL across a flexible joint interposed between the first body and a second body (operation 1212). The advancement may, for example, be accomplished with a plunger-like device, such as an IOL insertion tool similar to insertion tool 200. The flexible joint may, for example, provide a continuous surface between the inner surface of the first body and the inner surface of the second body.

Process 1200 additionally includes advancing the IOL through the second body to further fold the lens (operation 1216). The second body may, for example, have a tapering bore that facilitates the further folding.

Process 1200 also includes injecting an intraocular lens into an eye (operation 1220). The intraocular lens may, for example, be injected when it reaches the end of second body.

Although process 1200 illustrates one example of a process for using a system for intraocular lens surgery, other processes for using an IOL surgical system may include fewer, additional, and or a different arrangement of operations. For example, a process may not include receiving an IOL in a first body. The IOL may, for instance, have been received due to another process or have been shipped already inserted in the first body. As another example, a process may call for situating the IOL within an IOL surgical system. As a further example, a process may not call for injecting the IOL into an eye.

The various implementations discussed and mentioned herein have been used for illustrative purposes only. The implementations were chosen and described in order to explain the principles of the disclosure and the practical application and to allow those of ordinary skill in the art to understand the disclosure for various implementations with various modifications as are suited to the particular use contemplated. Thus, the actual physical configuration of components may vary. For example, the mentioned size(s) of components and their illustrated sizing relative to each other may vary based on application. Moreover, the shapes of one or more components may vary depending on application. Thus, the illustrative implementations should not be construed as defining the only physical size, shape, and relationship of components.

The terminology used herein is for the purpose of describing particular implementations only and is not intended to be limiting. As used herein, the singular form “a”, “an”, and “the” are intended to include the plural forms as well, unless the context clearly indicates otherwise. It will be further understood that the terms “comprises” and/or “comprising,” when used in the this specification, specify the presence of stated features, integers, steps, operations, elements, and/or components, but do not preclude the presence or addition of one or more other features, integers, steps, operations, elements, components, and/or groups therefore.

The corresponding structure, materials, acts, and equivalents of all means or steps plus function elements in the claims below are intended to include any structure, material, or act for performing the function in combination with other claimed elements as specifically claimed. The description of the present implementations has been presented for purposes of illustration and description, but is not intended to be exhaustive or limited to the implementations in the form disclosed. Many modifications and variations will be apparent to those of ordinary skill in the art without departing from the scope and spirit of the disclosure.

A number of implementations have been described for an intraocular lens surgical system and method, and several others have been mentioned or suggested. Moreover, those skilled in the art will readily recognize that a variety of additions, deletions, modifications, and substitutions may be made to these implementations while still providing an intraocular lens surgical system and method. Thus, the scope of the protected subject matter should be judged based on the following claims, which may capture one or more concepts of one or more implementations.

1. A system comprising:
a first body adapted to receive an intraocular lens;
a second body comprising a passage adapted to facilitate folding the intraocular lens as the lens is advanced through the passage; and
a flexible joint interposed in a gap between the first body and the second body.

2. The system of claim 1, wherein the first body is adapted to facilitate static folding of the intraocular lens.

3. The system of claim 1, wherein the first body comprises a first material, wherein the second body comprises a second material, and wherein the first material is more flexible than the second material.

4. The system of claim 1, wherein the first body comprises:
an open channel;
a first tab coupled to first end of the open channel; and
a second tab coupled to a second end of the open channel.

5. The system of claim 2, wherein the static folding of the first body does not materially distort the second body.

6. The system of claim 1, wherein the flexible joint comprises a flexible material molded over the first body and the second body.

7. The system of claim 6, wherein the flexible material is continuous from the first body to the second body.

8. The system of claim 5, wherein an inner surface defined by the flexible joint is substantially flush with an inner surface of the first body and an inner surface of the second body.

9. The system of claim 1, wherein the second body comprises a fluoropolymer.

10. The system of claim 1, wherein at least one of the first body or the second body comprises a first bonding element, wherein the flexible joint comprises a second bonding element, and wherein the first bonding element and the second bonding element cooperatively join the at least one of the first body or the second body and the flexible joint.

11. The system of claim 10, wherein the first bonding element comprises an annular groove, and wherein the second bonding element comprises a lip received into the annular groove.

12. The system of claim 10, wherein the first bonding element comprises a roughened surface, and wherein the second bonding element comprises a corresponding roughened surface.

13. The system of claim 10, wherein the first bonding element is a plurality of slots formed in an end surface, and wherein the second bonding element comprises a plurality of protrusions that are received into the plurality of slots.

14. The system of claim 10, wherein the first bonding element is a plurality of openings, and wherein the second bonding element comprises a plurality of protrusions received into the plurality of openings.

15. A method comprising:
statically folding an intraocular lens with a first body;
advancing the intraocular lens across a flexible joint interposed in a gap between the first body and a second body; and
advancing the intraocular lens through a passage in the second body to further fold the lens.

16. The method of claim 15, further comprising depositing the intraocular lens in the first body.

17. The method of claim 15, wherein statically folding an intraocular lens with a first body comprises moving a first tab coupled to one end of an open channel towards a second tab coupled to a second end of the open channel.

18. The method of claim 15, wherein the first body being comprises a first material, wherein the second body comprises a second material, and wherein the first material is more flexible than the second material.

19. The method of claim 15, wherein the static folding of the first body does not materially distort the second body.

20. The method of claim 15, further comprising injecting the lens into an eye.
